(19) United States
(12) Patent Application Publication
(10) Pub. No.: US 2018/0085152 A1
(43) Pub. Date: Mar. 29, 2018

(54) INTERTROCHANTERIC FIXATION DEVICE

(71) Applicants: FIROOZEH MADADI, TEHRAN (IR); FIROOZ MADADI, TEHRAN (IR)

(72) Inventors: FIROOZEH MADADI, TEHRAN (IR); FIROOZ MADADI, TEHRAN (IR)

(21) Appl. No.: 15/521,483

(22) PCT Filed: Oct. 19, 2015
(86) PCT No.: PCT/IB2015/058023

§ 371 Date: Nov. 22, 2017

(30) Foreign Application Priority Data
Oct. 24, 2014 (IR) 13935014000300813

(51) Int. Cl. A61B 17/74

(52) U.S. Cl. CPC A61B 17/742; A61F 2/32

(57) ABSTRACT

An intertrochanteric fracture fixation device. The device comprises an anterior an anterior right lateral quadrangular shaft, a posterior quadrangular shaft, an anterior left medial hemi quadrangular shaft, a plurality of screws and a plurality of rods. The vertical arm of anterior right lateral quadrangular shaft enters into the vertical arm of the posterior quadrangular shaft and the horizontal arm of the anterior left quadrangular shaft is inserted into the horizontal arm of the posterior quadrangular shaft. A plurality of spikes is present on both the combined parts of the device. The distance between each spike is 20 mm. The three shafts i.e. anterior right lateral quadrangular shaft, a posterior quadrangular shaft and an anterior left medial hemi quadrangular shaft are combined and fixed to the proximal part of the fractured site. Again the three shafts are combined and fixed to the distal part of the fractured site of the bone.

### BACKGROUND OF THE INVENTION

### Field of Invention

the embodiments disclosed herein relate generally to surgical devices and, more particularly, relates to an intertrochanteric bone fixation device.

### Description of the Related Art

The surgical devices and kits are involved in performing femoral neck and femoral shaft junction's fractures or intertrochanteric area. Intertrochanteric fractures are a common occurrence in the elderly people. Femoral fractures lead to functional impairments, increased morbidity and mortality in elderly people. Over a quarter of million hospital admissions among the elderly are due to Intertrochanteric fractures. The rates of the Intertrochanteric fractures are increasing day by day and will increase by 12% by 2030. Intertrochanteric fracture rates are highest in United States and Europe, intermediate rates are reported in Asian countries such as Iran, China and Hong Kong. Current treatment options includes hip screws and hemiarthroplasty, intertrochanteric fracture treatment options include placement of compression hip screw or intramedullary nails (IM Nail). Other option intertrochanteric fracture treatment includes the use of DHS, Jewett, Richards, blade plate or (PFNA) but generally outcome of these devices are moderately poor.

Reasons of failures are related to soft tissue and muscles debility and osteoporosis and physical inactivity. Therefore, switch from internal fixation to arthroplasty is a proper solution to the problem. The challenge in arthroplasty is to preserve the honey attachment of abductor and iliopsoas muscles. The present invention secures the fractured parts using intertrochanteric bone fixation that allows healing and prevent further fracture sequel.

Therefore, there is a need to develop an intertrochanteric bone fixation device in order to preserve proximal portion of a fractured bone, conserve the iliopsoas and abductor muscles of the hip and bone.

### BRIEF SUMMARY OF THE INVENTION

The invention overcomes the above described problem by introducing an intertrochanteric bone fixation device. The intertrochanteric bone fixation device reduces hypersensitivity reactions and other rejections. The advantage of using the intertrochanteric bone fixation device is the firm attachment that allows the reduction in healing time, muscular disturbance and further trauma to the bone. Therefore the intertrochanteric bone fixation device improves the treatment outcomes and reduces complications.

In view of the foregoing, an embodiment herein provides an intertrochanteric fixation device. An embodiment herein provides an intertrochanteric bone fixation device. The device comprises an anterior right lateral quadrangular shaft with right angle port and a plurality of spikes on the interior region, a posterior quadrangular shaft with right angle port and a plurality of spikes on bone contact interior wall, an anterior left medial hemi quadrangular shaft with hallow interior, a plurality of rods and a plurality of screws. The vertical arm of anterior right lateral quadrangular shaft enters into the vertical arm of the posterior quadrangular shaft and the horizontal arm of the anterior left medial hemi quadrangular shaft is inserted into the horizontal arm of the posterior quadrangular shaft.

In one of the embodiment, the device is made up of stainless steel 348L (316L) with chrome-cobalt coverage or titanium. In another embodiment, a plurality of spikes are present on all three parts of the device, wherein the base of each spike is 2 mm and height is 2.5 mm, wherein the distance between each spike is 20 mm. In another embodiment, the anterior right lateral shaft comprises of horizontal arm of length 35 mm and thickness of 5 mm, wherein the vertical arm of the anterior right lateral shaft is 55 mm with 3 mm diameter.

In one of the embodiment, the anterior left medial hemi shaft comprises a vertical arm of 30 mm with diameter 5 mm, wherein the length of the horizontal arm is 50 mm with 5 mm diameter. In another embodiment, the posterior quadrangular shaft comprises a vertical arm of 50 mm and a horizontal arm of 30 mm. In an embodiment, the three quadrangular shafts are assembled and is fixed to the proximal part of the fractured site, and again the three quadrangular parts are assembled and is fixed to the distal part of the fractured site.

In one of the embodiment, the rod connects the distal part to proximal part of the three quadrangular shafts by using a plurality of screws.

### BRIEF DESCRIPTION OF THE SEVERAL VIEWS OF THE DRAWINGS

FIG. 1 (a): Illustrate a preliminary design that has been further developed for optimal design and functionality. The part is only history of the present invention and is not used in surgery. The final design comprises of two identical proximal and distal segments that are connected at 3 loci on each right angle port and secured using screws such as Herbert, pins or spring system.

FIG. 1 (b): Illustrate a brushed interior wall of preliminary design;

FIG. 1 (c): Illustrate perspective view of a preliminary design;

FIG. 2 (a): Illustrate a perspective view of a posterior quadrangular shaft;

FIG. 2 (b): Illustrate a brushed interior wall of the posterior quadrangular shaft;

FIG. 2 (c): Illustrate a perspective view of a posterior quadrangular shaft;

FIG. 2 (d): Illustrate a perspective view of a posterior quadrangular shaft consists of 50 mm vertical shaft and 30 mm horizontal shaft. The two parts have a circular barrel interior for the introduction of FIGS. 3 (a) and 3 (b) on each side. A plurality of spikes is present on both the shafts and distance between each spike is 20 mm;

FIG. 3 (a): Illustrate a perspective view of an anterior right lateral quadrangular shaft;

FIG. 3 (b): Illustrate a brushed interior wall to provide a firm attachment;

FIG. 3(c): Illustrate a perspective view of anterior right lateral quadrangular shaft consists of 35 mm horizontal shaft and 55 mm vertical shaft that is 3 mm in diameter and enters into posterior quadrangular shaft. A plurality of spikes is present on the horizontal shaft.

FIG. 4 (a): Illustrate a perspective view of an anterior left medial hemi quadrangular shaft;

FIG. 4 (b): Illustrate a brushed interior wall to provide a firm attachment;

FIG. 4 (c): Illustrate a perspective view of anterior left medial hemi quadrangular shaft comprises a 50 mm horizontal shaft and 30 mm vertical. A plurality of spikes is present on the horizontal shaft.

FIG. 5: Illustrate a perspective view of an intertrochanteric bone fixation device installed on a fractured femur;

FIG. 6 (a): Illustrate a first part of the fixation device by combining three quadrangular shafts;

FIG. 6 (b): Illustrate a second part of the fixation device by combining three quadrangular shafts; and

FIG. 6 (c): Illustrate a combination of FIGS. 2 (a), 3 (a) and 4 (a).

### DETAILED DESCRIPTION OF THE INVENTION

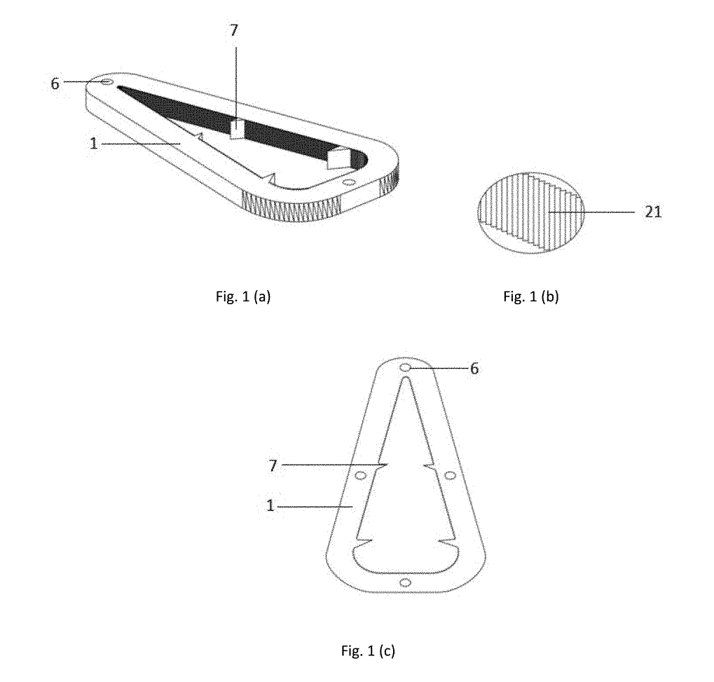

FIG. 1 (a) describes a preliminary design that has been further developed for optimal design and functionality. The preliminary design has a plurality of spikes (7), a hole (6) and a shaft (1). FIG. 1(b) describes a brushed interior wall (4) of the preliminary design. FIG. 1(c) describes a preliminary design.

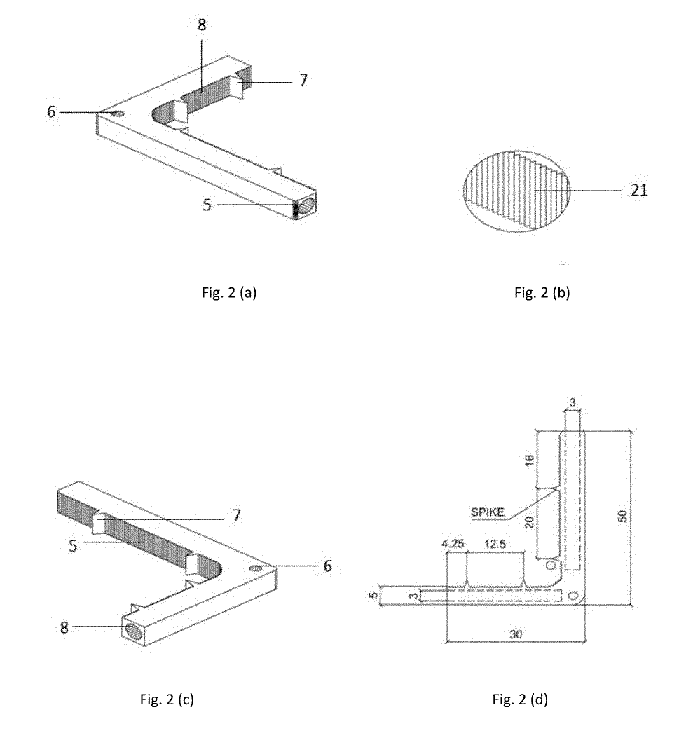

FIG. 2 (a) describes a perspective view of the posterior quadrangular shaft (31). The posterior quadrangular shaft (31) has a hollow vertical arm (5), a hollow horizontal arm (8) and a hole (6). Both horizontal arm and vertical arm comprises a plurality of spikes (7). FIG. 2(b) describes a brushed interior wall (21) of the posterior quadrangular shaft (31). FIGS. 2 (c) and 2 (d) describes a posterior quadrangular shaft (31). The height of the vertical arm (5) is 30 mm and the length of the horizontal arm (8) is 30 mm. The distance between each spike (7) is 20 mm.

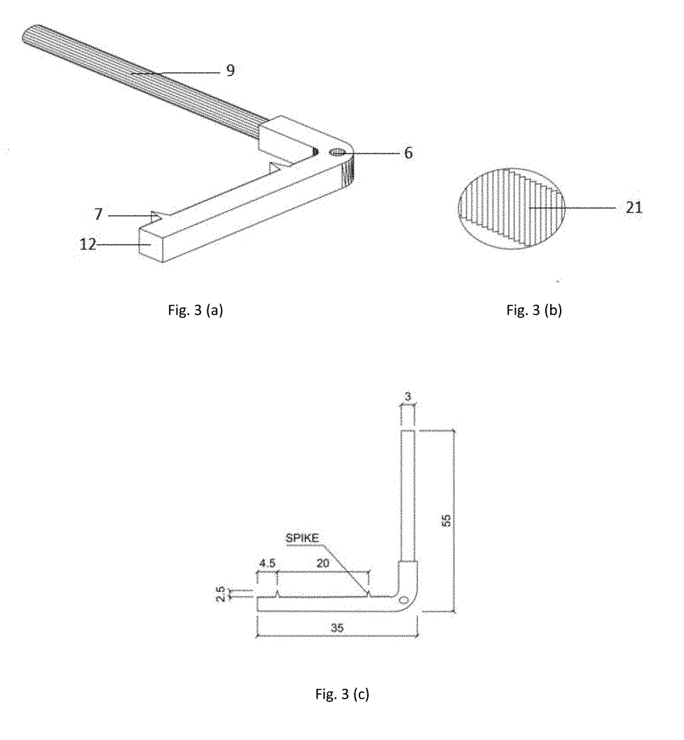

FIG. 3 (a) describes a perspective view of an anterior right lateral quadrangular shaft (33). The anterior right lateral quadrangular shaft (33) has a vertical arm (9), a horizontal arm (12) and a hole (6). FIG. 3 (b) describes a perspective view of a brushed interior wall (21) to provide a firm attachment. FIG. 3 (c) describes a perspective view of an anterior right lateral quadrangular shaft (33). The height of the vertical arm (9) is 55 mm and length of the horizontal arm (12) is 35 mm. The diameter of the vertical arm (9) is 3 mm. The vertical arm (9) of the right lateral quadrangular shaft (33) is inserted into the vertical arm (5) of the posterior quadrangular shaft (31). A plurality of spikes (7) is present on the horizontal arm (12) of the anterior right lateral quadrangular shaft (33).

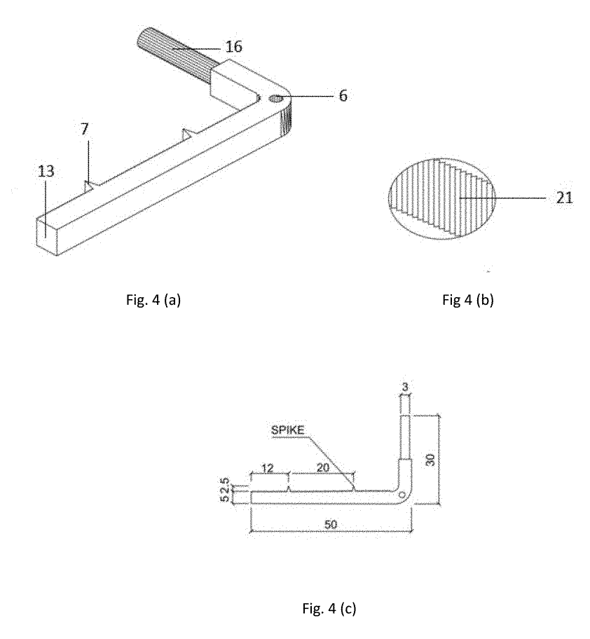

FIG. 4 (a) describes a perspective view of an anterior left medial hemi quadrangular shaft (35). The anterior left medial hemi quadrangular shaft (35) has a horizontal arm (16) and a vertical arm (13). FIG. 4 (b) describes a brushed interior wall (21) to provide a firm attachment. FIG. 4 (c) describes a perspective view of anterior left medial hemi quadrangular shaft (35). The vertical arm (13) of the anterior left medial hemi quadrangular shaft (35) is 30 mm and the horizontal arm (16) is 50 mm. A plurality of spikes (7) is present on the vertical arm (13) of the anterior quadrangular left medial hemi shaft (35). The distance between each spike (7) is 20 mm.

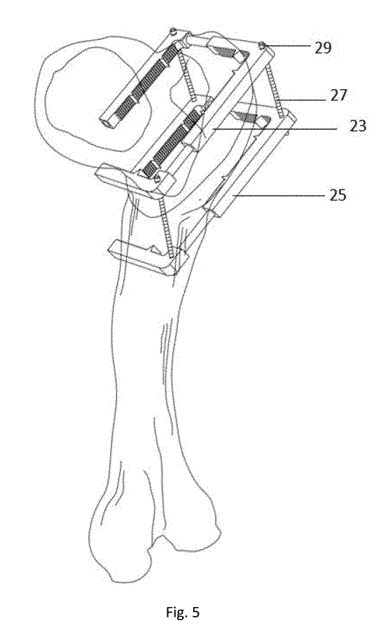

FIG. 5 describes a perspective view of an exemplary compliant intertrochanteric bone fixation device installed on a fractured femur. The device is fixed to the fractured part of the bone. The three parts i.e. posterior quadrangular shaft (31), anterior right lateral quadrangular shaft (33) and anterior left medial hemi quadrangular shaft (35) are combined together to form a first part (23) of the intertrochanteric bone fixation device. The first part (23) of the device is fixed to the proximal part of the fractured site. Again the three parts are combined to form a second part (25) and similarly the second part (25) of the three shafts is fixed to the distal part of the fractured site. The two combined parts are connects together by a plurality of rods (27) by using a plurality of screws (29).

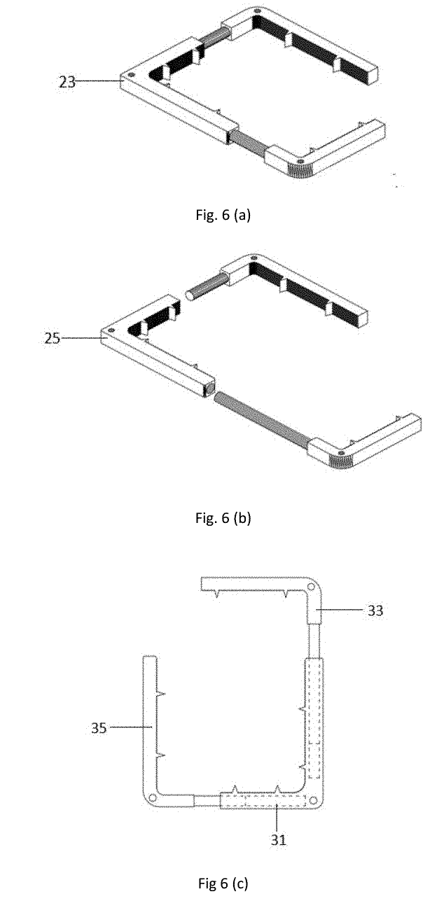

FIG. 6 (a) describes a first part (23) of the fixation device by combining three quadrangular shafts. FIG. 6 (b) describes a second part (25) of the fixation device by combining three quadrangular shafts. FIG. 6 (c) describes a combination of posterior quadrangular shaft (31), anterior right lateral quadrangular shaft (33) and anterior left medial hemi quadrangular shaft (35). The vertical arm (9) of the anterior right lateral quadrangular shaft (33) is inserted into the vertical arm (5) of the posterior quadrangular shaft (31). The horizontal arm (16) of the anterior left medial hemi quadrangular shaft (35) is inserted into the horizontal arm (8) of the posterior quadrangular shaft (31).

Construction material comprises of stainless steel 348L (316L) with chrome-cobalt coverage or titanium to reduce rejection or hypersensitivity reactions. Device is used under aseptic techniques and sterile. Usage of dynamic hip screws poses the risk of femoral head penetration by screws, fracture of femoral cortex, necrosis of femoral head and varus hip deformity (coxa vara). In order to prevent these problems preservation of the existing bone and muscles is important. Usage of intertrochanteric fixation device as a temporary or permanent treatment option helps to reduce muscle tone and prevent complications associated with screw and IM nails. Intertrochanteric device minimizes disruption to bone, has reduced tissue disturbance and postop pain and rehab potential.

The fracture is reduced as by longitudinal traction, internal rotation and adduction. Sagging of fracture site is corrected prior to intertrochanteric fixation. Skin incision of 4-6 centimeter is required distally at the tip of the greater trochanter. After meticulous open reduction of fracture parts, the intertrochanteric bone fixation device is inserted to the proximal part and distal parts of the fracture site in order to achieve a sequre grip externally by using a plurality of screws.

Then by power saw a cut is made on the femoral neck on the classic way and evacuate it, In case acetabulum is healthy then hemiarthroplasty is done. In case acetabulum is healthy then total hip arthroplasty is done. Then by reaming of proximal shaft and decision making for cement or uncemented femoral stem should fix, both, femoral fracture site and do arthroplasty to solve the problem of intertrochanteric fracture basically.

1. An intertrochanteric fracture fixation device, the device comprising:
an anterior right lateral quadrangular shaft; two spikes on the interior region;
a posterior quadrangular shaft;
an anterior left medial hemi quadrangular shaft;
a plurality of rods; and
a plurality of screws,
wherein the vertical arm of anterior right lateral quadrangular shaft enters into the vertical arm of the posterior quadrangular shaft and the horizontal arm of the anterior left quadrangular shaft is inserted into the horizontal arm of the posterior quadrangular shaft.

2. The device as claimed in claim 1, wherein the device is made up of stainless steel 348L (316L) with chronic-cobalt coverage or titanium.

3. The device as claimed in claim 1, wherein a plurality of spikes are present on all three shafts of the device, wherein base of each spike is 2 mm and height is 2.5 mm, wherein the distance between each spike is 20 mm.

4. The device as claimed in claim 1, wherein the anterior right lateral shaft comprises of a horizontal arm length of 35 mm with a diameter of 5 mm, wherein a vertical arm of the anterior right lateral segment is 55 mm with 3 mm diameter.

5. The device as claimed in claim 1 wherein the anterior left medial hemi quadrangular shaft comprises a vertical arm of 30 mm with diameter of 5 mm, wherein the length of a horizontal arm is 50 mm with 5 mm diameter.

6. The device as claimed in claim 1, wherein the posterior quadrangular shaft comprises a vertical arm of 50 mm and a horizontal arm of length 30 mm.

7. The device as claimed in claim 1, wherein the first part of the three quadrangular shafts is assembled and is fixed to a proximal part of the fractured site, and again a second part of the three quadrangular shafts is assembled and is fixed to a distal part of the fractured site.

8. The device as claimed in claim 1, wherein the proximal part and the distal part are connected by using the plurality of rod and the plurality of screws.
